(19) United States
(12) Patent Application Publication
(10) Pub. No.: US 2019/0257822 A1
(43) Pub. Date: Aug. 22, 2019

(54) SECURE MACHINE READABLE CODE-EMBEDDED DIAGNOSTIC TEST

(71) Applicant: PragmaDx, Inc., Nashville, TN (US)

(72) Inventor: Thomas F. Scherr, Nashville, TN (US)

(21) Appl. No.: 16/279,016

(22) Filed: Feb. 19, 2019

Related U.S. Application Data
(60) Provisional application No. 62/631,985, filed on Feb. 19, 2018.

(51) Int. Cl. G01N 33/53; G01N 33/52; G06K 19/06; G01N 33/563

(52) U.S. Cl. CPC G01N 33/5302; G01N 33/563; G06K 19/06037; G01N 33/523

(57) ABSTRACT

Disclosed herein is an information-augmented rapid diagnostic test in which control and test modules of a barcode, such as a QR code, are responsive to biomarkers in an analyte to become visible and form at least a portion of the barcode upon detection of the presence of such biomarkers. The barcode embeds test manufacturing details, serves as a trigger for image capture, enables registration for image analysis, and corrects for lighting effects. An accompanying mobile application preferably automatically captures an image of the test when the QR code is recognized, decodes the QR code, performs image processing to determine the concentration of the particular biomarker that is being diagnosed, and transmits the test results and QR code payload to a secure web portal.

### CROSS-REFERENCE TO RELATED APPLICATIONS

This application is based upon and claims the benefit of U.S. Provisional Patent Application No. 62/631,985 filed with the U.S. Patent and Trademark Office on Feb. 19, 2018 and titled “Machine Readable Code-Embedded Diagnostic Test,” which application is incorporated herein by reference in its entirety.

### FIELD OF THE INVENTION

The present invention relates generally to diagnostic tests, and more particularly to secure rapid diagnostic tests incorporating machine-readable codes.

### BACKGROUND OF THE INVENTION

There is extensive need for easily implemented tools for detecting the presence of certain elements in a sample, such as biomarkers, pathogens, and the like in a patient, contaminants in water and food supplies, etc. Of particular need are tools for quickly detecting the presence of certain analytes in biological samples of patients where quick and easy-to-use diagnostic tools are critical to controlling disease progression and individual patient outcomes. For example, as evidenced by a 21% decrease in case incidence between 2010 and 2015, recent malaria control efforts have been remarkably successful in lowering the burden that disease endemic countries face. Nonetheless, malaria led to 429,000 deaths in 2015 and 3.3 billion people remain at risk of infection. Plasmodium falciparum, the most prevalent form of the parasite, resulted in the deaths of 303,000 children under five years of age in 2015. Currently, treatment decisions are often symptom-based—which is confounded due to the overlap in clinical presentation between malaria and other tropical diseases. The diagnosis and treatment on the basis of symptoms alone results in a delay in diagnosis of nonmalarial causes of fevers, wasted resources, and potentially accelerated development of parasite resistance to treatment. Until there is a reliable vaccine against malaria, there is a need for improved diagnostic technologies to support elimination campaigns.

Further, the standard for clinical malaria diagnosis remains microscopic inspection of a blood smear. When performed by a trained technician, it can provide an accurate diagnosis within an hour. However, successful implementation remains difficult in environments that lack the needed infrastructure. Similarly, fluorescence staining and nucleic acid amplification through polymerase chain reaction are difficult to deploy in the regions most burdened by malaria.

Rapid diagnostic tests (RDTs) have been adapted as an attractive solution to the challenges presented in attempting fast diagnosis in low resource settings. RDTs can provide an inexpensive and fast diagnosis, and are amenable for use by minimally trained healthcare workers. Often in this approach, the tests take the form factor of a lateral flow assay (LFA).

Briefly, a blood sample is placed on the RDT sample pad, a running buffer is added to a buffer pad, and the blood flows through a porous membrane by capillary action. The accumulation of conjugate (typically gold nanoparticles conjugated to antibodies specific for a particular biomarker, such as in the exemplary case discussed above a malarial biomarker) results from a sandwich type assay at the “test” line, and confirms a positive diagnosis. Further downstream at the “control” line, a secondary antibody against the antibody on the conjugate captures any unbound conjugate. The presence of a control line signal indicates that the sample has successfully flowed through the test region and that sample conditions are satisfactory for antigen-antibody binding. If the control line is visible, the RDT result is considered valid.

Despite the mature biochemistry of RDTs, several issues still hinder their usage. Field evaluations indicate that correct analysis of RDTs is highly dependent on end-user training and supervision. Coupled with language barriers that lead to a poor understanding of operational instructions and errors in the interpretation of the results, RDTs often still result in an uncertain diagnosis. Additionally, there are currently no standards to evaluate field performance of RDTs, and lot-to-lot variation can result in misdiagnoses with the potential for harmful consequences. To combat these problems, the World Health Organization performs standardized testing of RDT performance which includes product testing, lot-testing, and testing at the end-user level. Others have recommended that RDT brand, lot number, and test conditions be recorded with each test and reported as feedback to the manufacturer. This would enable lot-to-lot comparisons, as well as validate satisfactory test storage conditions. Unfortunately, current reporting that describes the clinical usage of RDTs (such as malarial RDTs), as well as research publications, do not standardize this level of detail as a requirement, and as a result this potentially valuable information is lost.

In the exemplary case of malaria, many countries are shifting their efforts from malaria control to disease elimination. New technologies will be necessary to meet the more stringent demands of elimination campaigns, including improved quality control of diagnostic tests, as well as an improved means for communicating test results among field healthcare workers, test manufacturers, and national ministries of health, all while maintaining the data security and privacy of such information.

In other applications of rapid diagnostic tests, field healthcare workers are not necessary and patients perform the tests themselves. In these instances, a straightforward user workflow is necessary, and automated test analysis should be implemented in order to reduce misinterpretations of results. Furthermore, patient confidentiality will be a high priority, particularly for sensitive healthcare conditions.

### SUMMARY OF THE INVENTION

In accordance with certain aspects of an embodiment of the invention, disclosed herein is an embedded barcode incorporated in a rapid diagnostic test. This information-augmented diagnostic test operates on the familiar principles of traditional lateral flow assays, and in accordance with certain aspects of an embodiment of the invention, replaces the control line, and optionally the test line (or even multiple test lines) with a barcode, and more preferably with a control grid of individual modules patterned in the shape of a machine readable barcode, such as by way of non-limiting example a QR (quick response) code. In certain configurations, the test replaces the control line, and in certain configurations the test line, with only portions of such a QR code. In either case, after the test is processed, the complete QR code appears on both positive or negative tests. Likewise, the test line may be configured as in traditional lateral flow assays and positioned upstream of the QR code, or may alternatively be configured as one or more individual modules embodied within the grid that forms the QR code.

While exemplary embodiments described herein are set forth in the context of lateral flow assays, those skilled in the art will recognize that the principles of the invention may be implemented through other diagnostic formats, including by way of non-limiting example vertical flow-through, capillary flow based tests, milli- and microfluidic tests, and the like without departing from the spirit and scope of the invention.

In accordance with certain aspects of an embodiment of the invention, the multipurpose barcode described herein can be used not only to fulfill the control line role of test validation, but also to embed test manufacturing details, serve as a trigger for image capture, enable registration for image analysis, and correct for lighting effects. An accompanying mobile phone application preferably automatically captures an image of the test when the QR code is recognized, decodes the QR code, performs image processing to determine the presence and/or concentration of the particular biomarker that is being diagnosed, such as in the exemplary case discussed above, the concentration of malarial biomarker histidine-rich protein 2 at the test line or test modules of the QR code, and transmits the test results and QR code payload (including manufacturing information relating to the specific test) to a secure web portal. This approach blends automated, sub-nanomolar biomarker detection, with near real-time reporting to provide quality assurance data that will help to achieve malaria elimination.

The system and method set forth herein provide new and improved technologies to overcome one of the challenges in RDT analysis, specifically subjective visual inspection of RDTs. The system and method set forth herein apply image processing algorithms that analyze an image of an RDT captured with an imaging device, and more preferably a portable imaging device such as a camera on a mobile phone, to automatically interpret the test without any additional adapters or hardware. The “connected” nature of mobile phones also fulfills a major communication need for disease (e.g., malaria) elimination campaigns, which are often based on paper registries for disease surveillance. Given the growth of mobile phone usage, even in low- and middle-income countries, strategies that utilize a mobile phone have the potential to provide widespread deployment, even in remote point-of-care settings.

Disclosed herein is a strategy for including and retrieving additional test information, which is referred to herein as barcode embedded Rapid Diagnostic Tests (beRDTs). These diagnostic tests operate on the same principles as traditional lateral flow assays, except that the control line is replaced by a machine readable barcode, and more preferably a standard QR code to embed additional information. QR codes are built from individual “modules” comprising dark and light portions of a grid, which modules encode an information payload, as well as form patterns that assist in decoding the payload (FIG. 1(a)). QR codes offer unique advantages including a high information density per area and error correction of up to 30% of missing or defective modules. Due in part to their multi-platform readability and the diversity of the information that can be encoded (e.g., text, contact information, web URLs), static QR codes have previously been used to encode data in point-of-care diagnostic devices, but to date and to the inventors' knowledge have not had connection to the interpretation of the results. In accordance with certain aspects of an embodiment, the traditional control line is replaced on an RDT with a control-QR code (FIG. 1(b)).

The use of QR codes in accordance with aspects of an embodiment of the invention may provide certain specific features that may assist with disease elimination campaigns, such as by way of non-limiting example malaria elimination campaigns. The QR code configured and used as described herein may facilitate accurate quantitative image processing in two specific ways. First, the QR code allows for straightforward image registration, orientation, and scaling. The presence of a machine readable QR code simplifies the algorithmic image processing required to determine the location of the test line or test modules within the QR code. QR codes adhere to a strict standard that, among other things, includes modules forming specific patterns for machine image recognition (referred to herein as “machine read modules”) and requirements for contrast between dark and light modules. Importantly, the QR codes are present in the event of either positive or negative tests, as long as the gold conjugate and buffer have traveled the entire length of the test. This offers an unambiguous invalid test criterion. By using the successful recognition of a QR code as a trigger for a camera, we are assured that at the least, the minimal standard for machine recognition of QR codes has been met, which ensures that the image used for quantitative analysis of the test line and test modules within the QR code will be adequate. Upon code recognition, an image of the QR-control line and the test line or test modules are captured in a single image, and the embedded QR payload is parsed. Embedded within the QR code is manufacturing information, which manufacturing information may include (by way of non-limiting example) the test type, lot number, and expiration date (FIG. 1(a)). Finally, the test results and embedded information payload can be uploaded to any electronic health record system or electronic database. For example, a mobile application configured as described herein may be coupled with REDCap, a secure research database manager for analysis and server-side storage.

Moreover, maintaining the security of diagnostic test results can be quite important, particularly when testing for the presence of and ultimately diagnosing a patient's medical condition. To address that security need, and in those configurations in which the test modules are incorporated in the grid that forms the QR code, one or more available modules of that grid are randomly selected to serve as test modules that serve the same function as a test line on a traditional lateral flow assay device. In this configuration, data encoded into the QR code may be used to identify the X-Y coordinates of those test modules, and may require that a mobile device that has obtained an image of a test device configured as described herein communicate with a secure, remote data service to identify which of the modules that appear after carrying out a test are, in fact, test modules. Thus, a third party is prevented from determining which modules of the QR code are test modules, or what biomarkers they test for, without accessing such secure, remote data service. Such a configuration improves significantly upon prior efforts to perform rapid diagnosis through detection of biomarkers in a patient fluid sample.

In accordance with certain aspects of a particular embodiment, a system is provided for determining the presence of an analyte in a sample, comprising: a server computer having computer executable instructions stored thereon configured to: generate a barcode template including (i) machine read modules designating X-Y coordinates of a first group of modules necessary for enabling machine reading of the barcode with an imaging device, and (ii) manufacturing data modules designating X-Y coordinates of a second group of modules encoding manufacturing data relating to a rapid diagnostic test; and (iii) randomly select from the grid of modules X-Y coordinates of a third group of modules for use as test modules printed with a molecular recognition element specific to the analyte; a database in data communication with the server computer storing the barcode template; a rapid diagnostic test device comprising a substrate and a barcode printed on the substrate, the barcode comprised of machine read modules printed at the X-Y coordinates of the first group of modules, manufacturing data modules printed at the X-Y coordinates of the second group of modules, and test modules printed at the X-Y coordinates of the third group of modules; and a mobile computer software application executable on a mobile computing and imaging device, the mobile computer software application including computer instructions to: capture an image of the barcode on the rapid diagnostic test device; transmit the image of the barcode to the server computer to cause the server computer to analyze the X-Y coordinates of the third group of modules to detect the presence of the analyte; and receive from the server computer a diagnosis of the presence of the analyte.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized. The present invention is illustrated by way of example, and not by way of limitation, in the figures of the accompanying drawings, in which like reference numerals refer to similar elements, and in which:

FIG. 1(a) provides a schematic view of the components of a QR code in accordance with certain aspects of an embodiment of the invention.

FIG. 1(b) shows a schematic view of a barcode, and more particularly a QR code on a test device in accordance with certain aspects of an embodiment of the invention.

FIG. 2(a) shows a static rapid diagnostic test formed in accordance with certain aspects of an embodiment of the invention.

FIG. 2(b) is a chart of pixel intensity across a series of consecutive modules on the rapid diagnostic test of FIG. 2(a).

FIG. 3(a) shows an exemplary rapid diagnostic test according to certain features of an embodiment of the invention.

FIGS. 3(b) and 3(c) show graphs of pixel intensity based on volume of conjugate added and antibody concentration, respectively, on the rapid diagnostic test of FIG. 3(a).

FIG. 4(a) shows an exemplary rapid diagnostic test according to certain features of an embodiment of the invention.

FIG. 4(b) shows a graph of pixel intensity for different volumes of conjugate used on the rapid diagnostic test of FIG. 4(a).

FIGS. 5(a)-5(i) are various views of a rapid diagnostic test, a web browser user interface window showing uploaded data from a mobile user rapid diagnostic test imaging device, and various views of such a mobile user rapid diagnostic test imaging device through the process of scanning and analyzing a rapid diagnostic test, all according to certain features of an embodiment of the invention.

FIG. 6(a) shows exemplary images from a mobile imaging device of rapid diagnostic tests (with arrows indicating test line location) in accordance with certain aspects of an embodiment of the invention.

FIG. 6(b) shows a graph of integrated signal from the mobile image processing application generating the images for FIG. 6(b) for varying concentrations of rcHRP2.

FIG. 7 is a schematic view of a single rapid diagnostic test and exemplary user screens from mobile image capture and analysis software on a user's mobile device in accordance with certain aspects of an embodiment of the invention.

FIG. 8 is a schematic view of a rapid diagnostic test according to further aspects of an embodiment of the invention.

FIG. 9 is a schematic view of exemplary user screens from mobile image capture and analysis software on a user's mobile device in accordance with still further aspects of an embodiment of the invention.

FIG. 10 is a schematic view of a software architecture for manufacturing and analyzing rapid diagnostic tests in accordance with still further aspects of an embodiment of the invention.

FIG. 11 is a schematic view of various exemplary images produced from the mobile software in accordance with certain aspects of an embodiment of the invention.

FIGS. 12(a) and 12(b) are schematic views of lateral flow assays showing the effect on colorimetric intensity of varied placement of test modules on the membrane of the test device.

### DETAILED DESCRIPTION OF THE PREFERRED EMBODIMENTS

The invention may be understood by referring to the following description, claims, and accompanying drawings. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

Diagnostic tests employing a machine readable barcode, and more particularly a QR code, in accordance with certain aspects of an embodiment were configured following experiments designed to: 1) evaluate the QR code spatial resolution after fabrication, 2) determine the sensitivity of QR code pixel intensity to fabrication parameters, and 3) calculate the limit of detection of beRDTs at the test line using an automated mobile application configured as described herein. The beRDTs that were used in these experiments can be classified into two categories: static and dynamic. Static beRDTs refer to the membranes in which a visible solution has been deposited in the locations of the QR code modules in order to evaluate fabrication resolution. Dynamic beRDTs refer to tests that have an antibody solution that is invisible after deposition and drying, but that becomes visible only when gold nanoparticle conjugate binds within the QR code. While static beRDTs are useful for the evaluation of reagent deposition and image analysis software development, only the dynamic beRDTs are reactive to a biological sample.

One of a variety of commercially available third-party barcode generation libraries may be used to translate the payload text into a QR code. Multiple commercially available barcode generation software programs are available to generate a list of spatial X-Y coordinates of the “dark” modules of the QR code that correspond to locations where antibody was deposited. Likewise, multiple reagent deposition platforms are readily commercially available that may be used to deposit antibody solution onto a nitrocellulose membrane at the specific locations provided by the barcode generation software program. In addition to the QR code, a series of contiguous droplets was deposited 2 mm upstream of the leading edge of the QR code to form the test line. As in traditional lateral flow assays, the deposited antibodies adsorb to the nitrocellulose surface at the location of their deposition and do not flow down the membrane when a sample is added. Since the different capture antibodies are spatially separated, there is no cross-reaction between the QR code antibody and the test line antibody. The deposited solution varied depending on the objective of the experiment: for visualization of static beRDTs, 2 mM Eosin-Y (#E4009, Sigma Aldrich, St. Louis, Mo.) was used; for dynamic beRDTs, a solution of 1 mg mL⁻¹ goat anti-mouse antibody (#31160, Life Technologies, Grand Island, N.Y.) in PBST (1× phosphate buffered saline, 0.1% Tween-20) were used to print the control QR codes and the test line was made from a solution of 1 mg mL⁻¹ mouse anti-HRP2 antibody (#ABMAL-0404, Arista Biological, Allentown, Pa.). The overall QR code dimensions are 12.5 mm wide by 12.5 mm long; and the test line is 12.5 mm wide by 0.5 mm thick.

In preparing the conjugate, gold nanoparticles (AuNPs) 40 nm in diameter were purchased from DCN Diagnostics (Carlsbad, Calif.) at a concentration that has an absorbance, or optical density (OD), of 2.12 and used to create the gold-antibody conjugate used for detection. In this work, OD measurements of gold conjugate were made at 535 nm using an Agilent 8453 G1103A spectrophotometer (Agilent Technologies, Santa Clara, Calif.). The stock AuNPs were diluted down to an OD of 1 using deionized water. The pH of the resulting solution was then increased to 7.46 using a 0.20 M potassium carbonate solution. Mouse anti-HRP2 antibody (#ABMAL-0404, Arista Biological) was added to this gold nanoparticle solution at a protein concentration of 0.01 mg mL⁻¹, and then incubated for 15 minutes on a shaker. Blocking buffer (50 mM borate buffer with 10% (w/v) bovine serum albumin (BSA)) was added at a volume of 10% of the solution volume and the solution was mixed on a shaker for 45 minutes. After incubation the solution was centrifuged for 30 minutes at 5000 rcf, the supernatant removed, and the remaining conjugate pellet was diluted to an OD of 10 with storage buffer (50 mM borate buffer with 1% (w/v) BSA).

Nitrocellulose membranes with a 10 mil polystyrene backing were used to construct the rapid diagnostic tests. The control QR code and test line were deposited onto the nitrocellulose membrane, as described above, and the membrane was dried at 40° C. Next, the membrane was blocked with 100 mL Pierce Protein-Free T20 blocking buffer (ThermoFisher, Waltham, Mass.) and dried at 40° C. A wicking pad was attached to the backing card with the wicking pad overlapping the nitrocellulose membrane. A membrane cutter was used to cut the assembled RDT into test strips that were slightly larger than the width of the QR code. RDTs were stored in foil pouches with desiccant until use. Prior to use, the backing card was cut to remove the sections for a sample pad and a conjugate pad, leaving a “half-strip”. Half-strip tests function as dipstick-like lateral flow assays and are common during the development of full RDTs. A paperclip was used to ensure contact between the wicking pad and the nitrocellulose membrane—a role played by the plastic housing in commercial RDTs. Exemplary dimensions of the beRDT are as follows: the nitrocellulose membrane of the beRDT is 19 mm wide by 24 mm long; the wicking pad is 19 mm wide by 19 mm long; the entire beRDT (membrane and wicking pad) is 19 mm wide by 40 mm long; there is 3 mm of overlap between the nitrocellulose membrane and the wicking pad. Some commercial rapid diagnostic tests include a cover over the membrane to protect from environmental contaminants, but many do not and recent efforts have shown that paper-based diagnostic devices are tolerant of surface contaminants. For simplicity of fabrication, we chose not to include a cover on the beRDTs in this work.

Experiments were then conducted with beRDT's assembled in accordance with the foregoing description. The assembled beRDTs were placed in a 3D printed holder with a 210 μL liquid reservoir. To evaluate the effect of deposited antibody concentration and conjugate volume on colorimetric intensity of the resulting QR code, varying amounts of conjugate were mixed with PBSTT running buffer (1×PBS, 0.1% Tween-20, 2% Triton-x-100), to a total solution volume of 200 μL. To determine the beRDT limit of detection, 110 μL of the PBSTT running buffer was pre-mixed with 40 μL of conjugate and 50 μL of 0-50 nM recombinant HRP2 (obtained from PATH, Seattle, Wash.) and added to the reservoir. In all cases, the assays were run until the running buffer had completely wicked from the liquid reservoir. Quantitative imaging was performed ˜45 minutes after the sample was added to the assay. A digital camera was used to image the parametric evaluation of QR code colorimetric intensity, and ImageJ (NIH, Bethesda, Md.) was used for quantitative image analysis. To determine the limit of detection, a mobile phone application was provided to perform automated image analysis and quantify the intensity of the test line. The limit of detection was calculated as described by Wang (IUPAC method).

The mobile application, Q-beRDT (Quantitative-barcode embedded Rapid Diagnostic Test), was written in the Swift programming language and deployed on an iPhone 6s (Apple, Cupertino, Calif.). Q-beRDT may employ readily commercially available barcode scanning software, such as by way of non-limiting example the Scandit Software Development Kit (SDK), to scan QR codes. In preliminary studies, several third-party barcode libraries as well as Apple's native framework were evaluated, and the Scandit SDK was found to be the most effective at recognizing low contrast QR codes. The mobile application initiates a scanning view and each consecutive frame from the camera is processed by the SDK in search of a machine-readable QR code. When a QR code is recognized, the Q-beRDT application used the dimensions of the QR code for reference and cropped the frame that detected the QR code to include the QR code, and in this particular test the upstream test line, although such software may likewise crop the image to solely the QR code in those configurations in which test modes are embedded within the QR code instead of a separate upstream test line. The data payload embedded within the QR code was decoded and displayed to the end-user along with the cropped image of the beRDT. A 1 mm wide linescan was made from the top of the image to the bottom and the average pixel intensity across each horizontal cross-section was calculated. Using the pixel intensity of the QR code (I_{QR}) as a constant dark value, and the pixel intensity of the nitrocellulose membrane (I_{NC}) as a constant light value, the pixel intensities (Iᵢ) in the line scan were normalized (I_{ĩ}) to account for variations in lighting (eqn (1)):

$${\overline{I}}_{i} = \frac{{I}_{i} - {I}_{QR}}{{I}_{QR} - {I}_{NC}}$$

The center linescan (1 pixel wide) was displayed to the user. Using the dimensions of the QR code for a size-scale, an algorithm is applied to find test line signal and integrate the area under the peak curve to calculate a test line signal. Finally, Q-beRDT used the REDCap, a web-based research database management platform, Application Programming Interface (API) to upload the following as a new REDCap record: sample ID; manufacturing information including test type, lot number, and expiration date; test line signal; and the cropped image of the beRDT. Importantly, except for entering a test identifier, the software is entirely automated. The simple interface guides the end-user through the software with the ability to go on to the next stage of beRDT analysis, or to re-do the previous step if desired. Q-beRDT only requires manual intervention to point the camera at the beRDT until it is recognized, and to enter a sample identification before uploading the results to REDCap.

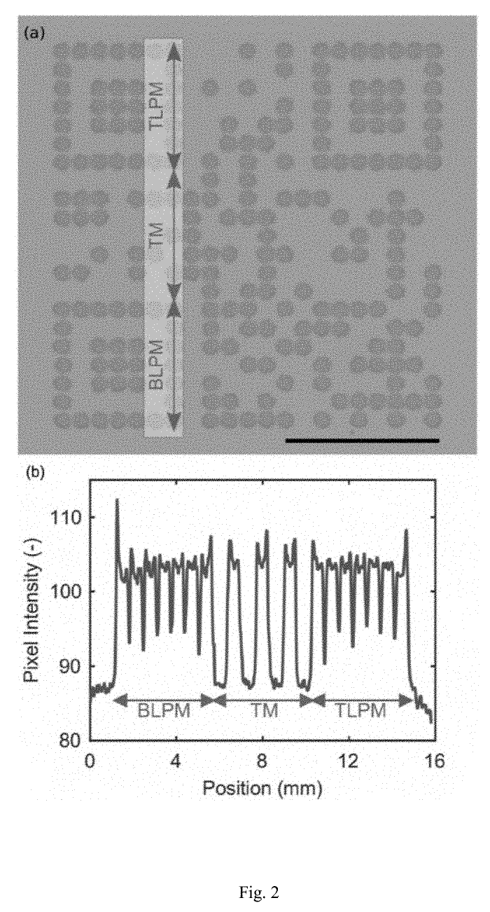

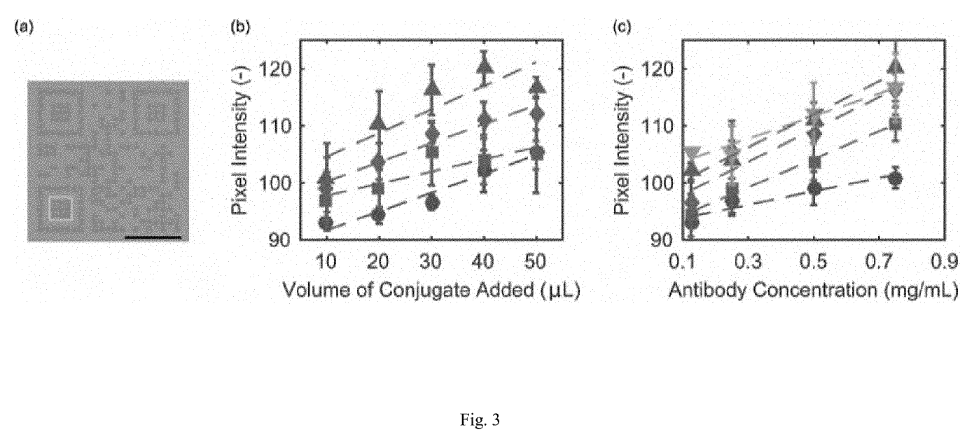

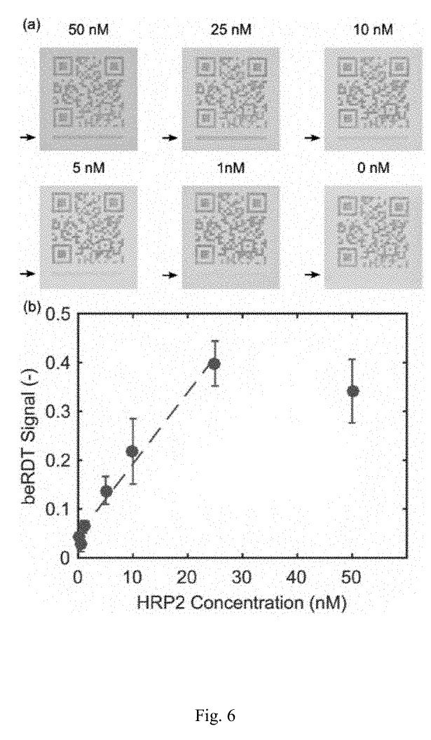

Detection of a machine-readable QR code relies on satisfying a set of standards. FIGS. 2-4 herein include annotations that emphasize particular features of the QR code that were used for experimental analysis. These annotations, while helpful in explanations of the results, will interfere with the requirements for necessary QR code recognition and lessen the likelihood of QR code scanning. In FIG. 6, there are no annotations and the QR codes of these beRDTs are machine readable. Those beRDTs have been tested with the freely available Scandit iOS mobile application (the same library utilized in the mobile phone application). Videos of successful scanning events of the QR codes in FIGS. 3-6 were obtained, as well as time-lapse videos of beRDT processing and a screen recording of the Q-beRDT software.

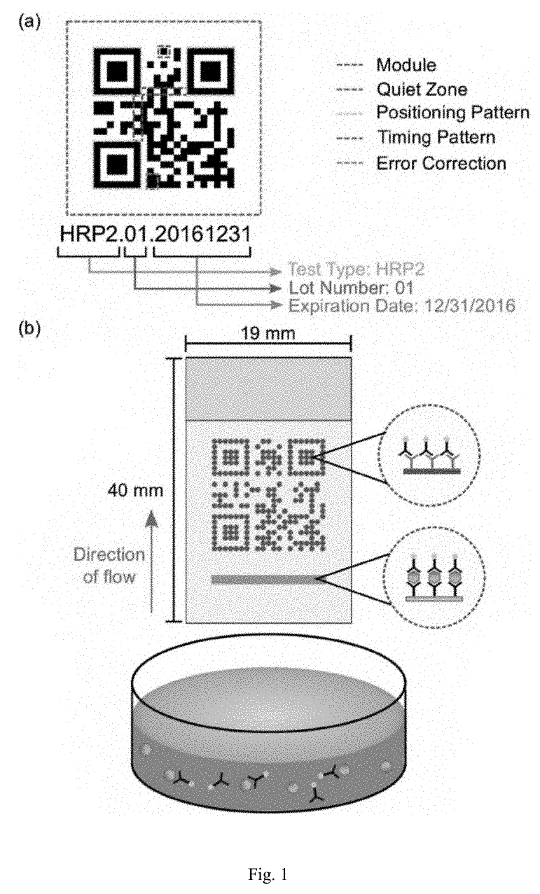

FIG. 1(b) shows a beRDT configured in accordance with certain aspects of an embodiment of the invention. In a halfstrip of dipstick form factor, beRDTs configured as described herein use the same principles and biochemistry of traditional lateral flow assays, but with at least the control antibodies, and preferably the test antibodies, deposited in the shape of a QR code.

Successful QR code capture requires that sufficient contrast between dark and light modules is maintained. In addition, overall size must be minimized to keep costs low and facilitate use at the point-of-care. A colorimetric dye, Eosin-Y, was directly deposited onto a nitrocellulose membrane to evaluate how the manufacturing parameters affected these desired outcomes. FIG. 2(a) shows the result of the optimized manufacturing parameters to produce a 12.5 mm by 12.5 mm QR code made from a 2 mM Eosin-Y solution (scale bar is 5 mm). Each dark module consists of a single 40 nL droplet of Eosin-Y, and creates a solid 0.55 mm diameter circle on the nitrocellulose membrane. The dark modules have distinct edges from one another, and are clearly visible on the white nitrocellulose membrane. Some surface “roughness”, where the perimeter of an individual module is slightly jagged, is visible as well. For some of the dark modules, there is a noticeable “coffee-ring” effect where the edges of the droplet are slightly darker in intensity than the center portion of the droplet. This is confirmed with a line scan of pixel intensity that follows along the bottom left positioning modules, the timing pattern, and the top left positioning modules (FIG. 2(b), providing a pixel intensity line scan across a series of consecutive modules, and in which BLPM=bottom left positioning modules, TM=timing modules, and TLPM=top left positioning modules). In the pixel intensity line scan, there are spikes in pixel intensity at the edges of the modules. Importantly, the surface roughness and coffee-ring effect do not impact the machine-readable nature of the QR code as evidenced by the rapid detection of the QR code.

Using a QR code in place of a control line requires that the QR code appears and can be recognized by a phone or other remote imaging device, regardless of the test outcome. There are several variables that affect the intensity of a signal on a rapid diagnostic test. We focused on one fabrication variable, the concentration of the deposited capture antibody, and one operational variable, the volume of detection antibody-gold nanoparticle conjugate added to the test. While a test device in accordance with certain aspects of an embodiment of the invention is described herein as employing such antibodies, optionally other molecular recognition elements (e.g., any reagent that can be used to capture or detect an analyte) may likewise be utilized without departing from the spirit and scope of the invention.

FIG. 3 shows pixel intensity within the inner portion of the bottom left positioning module pattern (yellow box of FIG. 3(a)), with goat anti-mouse IgG capture antibodies and mouse anti-HRP2 gold conjugate for detection with varying amounts of volume of conjugate added to the sample (FIG. 3(b)), and capture antibody concentration (FIG. 3(c)). With reference to FIG. 3(b), the antibody ink concentration is as follows: black circle=0.125 mg mL⁻¹; blue square=0.25 mg mL⁻¹; purple diamond=0.5 mg mL⁻¹; red triangle=0.75 mg mL⁻¹. Likewise, with reference to FIG. 3(c), conjugate volume added to the sample is as follows: black circle=10 μL; blue square=20 μL; purple diamond=30 μL; red triangle=40 μL; orange inverted triangle=50 μL. The scale bar in FIG. 3(a) is 5 mm. Increasing either the volume of conjugate added to the test or the capture antibody concentration of the QR code resulted in darker intensity modules (FIGS. 3(b) and (c)). At the lowest volume of conjugate, increasing the concentration of the antibody deposited eventually results in saturation at a relatively low pixel intensity. At such low volumes of conjugate added, most of the conjugate binds and the excess antibody remains unbound. In contrast, intensity saturation is seen when the conjugate is in large excess. Generally, as antibody concentration or volume of conjugate increase, the resulting average pixel intensity increase seen in the bottom left positioning modules is linear until saturation. It is envisaged that other parameters might affect QR code performance including membrane porosity, antibody selection, blocking conditions, and running buffer selection.

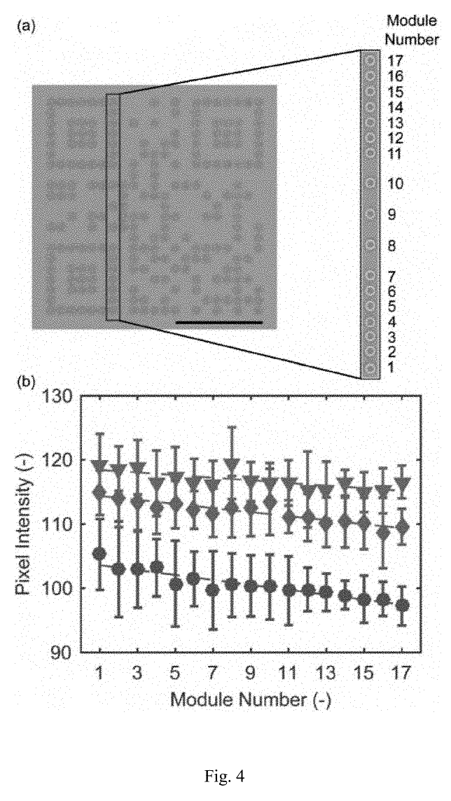

While average pixel intensity of a group of modules near the leading edge of the QR code is one metric to evaluate the colorimetric intensity, it does not take into account the potential for binding of conjugate in the upstream modules to affect the downstream modules. To evaluate this, the average pixel intensity of a series of consecutively downstream modules was analyzed along the bottom left positioning pattern, timing pattern, and top left positioning pattern (FIG. 4(a), showing the gradient of average pixel intensity of consecutive modules (yellow circles) in the bottom left positioning pattern, timing pattern, and top left positioning pattern, with the scale bar shown being 5 mm). In FIG. 4(b), the resulting change in intensity in downstream modules is shown for a constant capture antibody concentration at 0.5 mg mL⁻¹, for different volumes of conjugate added (50 μL purple inverted triangle; 30 μL blue diamond; 10 μL black square). With 10 μL of conjugate, there is less conjugate available for binding at each subsequent module as the sample moves downstream; this results in a lower module intensity for the downstream modules. At this volume, there is insufficient contrast between the dark and light modules for successful QR code recognition. This depletion effect is less pronounced with 30 μL of conjugate, and is negligible at 50 μL of conjugate. At high enough volumes, there is sufficient conjugate available for binding at the downstream sites to maintain a constant pixel intensity. With at least 30 μL of conjugate, the resulting beRDTs have QR codes that are uniform in pixel intensity and are dark enough that they are readily identified by the mobile phone application.

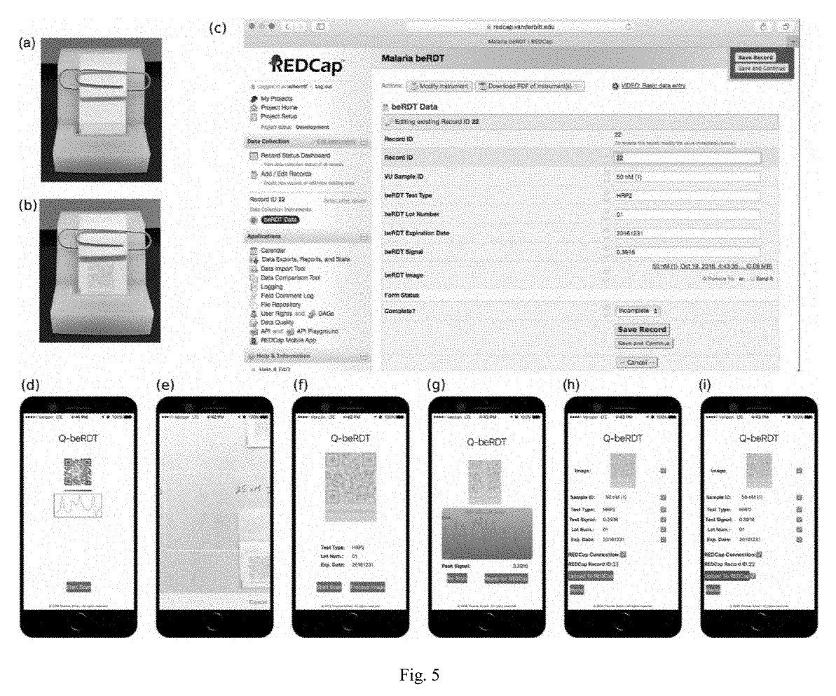

Using the above-identified conditions that resulted in uniform and machine readable QR codes, the limit of detection of the assays and the mobile-phone application were determined. Concentrations of HRP2 between 0-50 nM were added to a 3D-printed beRDT holder and sample reservoir (FIGS. 5(a) and (b), FIG. 5(a) showing the beRDT immediately after adding the beRDT, and FIG. 5(b) showing the beRDT 15 minutes after adding the test to 50 μL of 25 nM ITG, 40 μL of conjugate, and 100 μL of PBSTT), analyzed by the Q-beRDT software (FIG. 5(d)-(i), showing screen shots of the mobile application software that quantitatively analyzes beRDTs and sends the results to a data storage facility, such as by way of non-limiting example REDCap), and the data was linked to REDCap for storage (FIG. 5(c) showing the record uploaded from the mobile application software). The beRDT signal, the integrated area of the pixel intensity curve across the test line, was normalized to account for variations in lighting.

The signal linearly increased with increasing HRP2 concentration up to 25 nM after which the signal appeared to saturate (FIG. 6). FIG. 6(b) shows the integrated signal from the q-beRDT mobile phone application with varying concentrations of rcHRP2 (mean±standard deviation, n=3 for each concentration), with the dashed line comprising the linear fit of the data, excluding the 50 nM data points that are outside of the linear range of the test. The signal saturation, as well as the magnitude of the variation from test to test, are commonly seen in lateral flow assays. We determined the statistical limit of detection to be 0.966 nM. HRP2 production rates can vary among strains and developmental stage of the parasite, but previous work has reported correlations between HRP2 concentration and cultured Plasmodium falciparum parasite concentration. Using this conversion, our limit of detection can be approximated to be 543 parasites per μL.

In this process, we evaluated two parameters (capture antibody concentration and volume of conjugate) with the purpose of making reproducible and easily-detectable QR codes. Even with our focus on generating machine-readable beRDTs, our prototype has a limit of detection within the clinical spectrum of malaria infections. Under these test conditions, which were optimized to generate reproducible machine readable QR codes, this limit of detection falls between the high parasitemia values (2000 par μL−1 and 5000 par μL−1) and the low parasitemia value (200 par μL−1) that the WHO uses in benchmark testing for commercial RDTs. Further optimization to improve the limit of detection at the test line could include the evaluation of different antibodies and materials, conjugate fabrication conditions, as well as blocking and running buffers.

As described herein, by converting the control line on a rapid diagnostic test, such as a malaria rapid diagnostic test, into a mobile phone-readable barcode, such as a QR code, embedded with manufacturing details, we merge quality assurance, record keeping and reporting, and automated test line analysis into a single reporting channel. The control antibodies in the QR code assist with image processing, and also provide standardized test rejection criterion. This platform could readily be integrated into a variety of mobile health initiatives for malaria, and at least surveillance efforts for other infectious diseases.

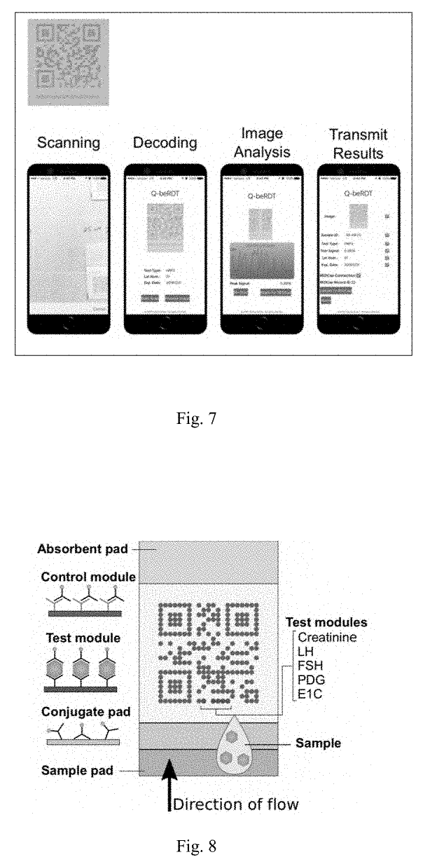

The foregoing describes the feasibility and general construction of a lateral flow assay with the traditional “control line” manipulated to take the shape of a two-dimensional barcode in accordance with certain aspects of an embodiment of the invention. In the foregoing configuration, the “test line”, which was upstream of the machine-readable barcode, served as a traditional test line: the target analyte was captured by immobilized molecular recognition elements, and detected by a signal nanoparticle that had complimentary molecular recognition elements functionalized to the surface. A summary schematic view of that overall platform (hardware and software) is shown in FIG. 7. In that configuration, a software component is also provided. The software consists of a mobile phone application, and server-side functionality. The mobile application uses a barcode scanning algorithm to identify a machine-readable barcode. Upon recognition, the application initiates the capture of a photograph of the test and decodes the information payload of the barcode. The mobile phone application uses custom digital image and signal processing to identify registration features of the barcode, and to find the location of the test line. The colorimetric intensity of the test line is quantified through these computer vision algorithms. The foregoing shows a quantitative relationship between the color intensity of the test line and the target analyte concentration.

The barcode information payload is parsed for information; in the embodiment described above, the barcode contains manufacturing information. This information, along with other information manually entered by the app-user, and the normalized (between light and dark sections of the test) colorimetric intensity of the test line, are transmitted to REDCap (a web-application for research data capture).

Importantly, beRDTs configured in accordance with an embodiment of the invention may have several advantages over traditional lateral flow assays, including one or more of the following: automated, objective, and quantitative analysis; straight-forward user-workflow; easy integration with electronic medical records; and readily facilitated integration between end-use and manufacturing.

In accordance with further aspects of an embodiment of the invention, and with particular reference to FIG. 8, an exemplary beRDT for fertility hormone measurement and analysis is provided. This configuration includes multiplexed biomarker detection, specifically identifying four target hormones. It also includes the measurement of another biomarker, creatinine, as a control for sample volume/dilution factors. Rather than an explicit test line, the capture reagents are directly incorporated as test modules of the barcode. Using methods described in greater detail below, computer image processing software uses the registration features of the barcode to identify the test module locations, and quantifies their colorimetric intensity accordingly as described above. Importantly, the computer image processing software accounts for rotation and perspective, as well as scale. In certain configurations, the remainder of the barcode is made of control modules that appear regardless of the presence of a biomarker. In other configurations, only a portion of the remainder of the barcode is made of such control modules, with the balance of the remainder of the barcode being printed with non-reactive colorimetric reagents that adsorb to the test device nitrocellulose membrane, as discussed in greater detail below.

Software is provided and accessible from a remote service, such as a remote server or Internet-hosted application, that determines suitable locations of test modules: whether it is taking an “off”/“light” module and making it “on”/“dark”, or vice-versa. Barcodes, two-dimensional barcodes in particular, have certain patterns that must appear in order to maintain the ISO standards that are used to decode them. As used herein, the term “machine read modules” is intended to refer to modules of a grid forming a barcode on a test device according to certain aspects of the invention that particularly form such patterns to maintain readability. When modifying existing modules of a barcode, it is important to avoid modifications to such machine read modules that will render the machine-readable barcode no longer readable (for example: the quiet zone, the timing pattern, the position and alignment patterns, the format and version information modules). To this end, software is provided that identifies barcode modules that can be modified, and modules that should not be altered, so as to keep the barcode machine-readable. This software provides utility in an automated manufacturing setting where the barcode patterns will change with each new test manufactured, and it is not feasible for manual selection of the appropriate modules of a barcode to modify.

Also provided is the necessary server-side software for full database integration. This includes the entire life-cycle of the test: manufacturing, sales, and field-use. This software couples the mobile application with a server-side database through an application programming interface. The database links together the particular test through its test identification number, lot number, and batch number. This information is then tagged to a particular end-user through sales tracking or other registration methods. Finally, upon use by the end-user, the database is updated with the results of the test. This integration between manufacturing, sales, and usage enables quality control/assurance efforts. For instance, in the event that a batch of tests is identified to have a manufacturing defect, the database can alert either the point-of-sales or the consumer of the potential for a defective test—an important concern for healthcare diagnostic testing. In addition, the database can collect the following information from the mobile app (using the built application programming interface): patient identifiers, login credentials, patient demographics, patient symptoms, barcode embedded test results, and clinical recommendations. It can also use this information as inputs into machine-learning algorithms or other artificial intelligence/data analytics efforts.

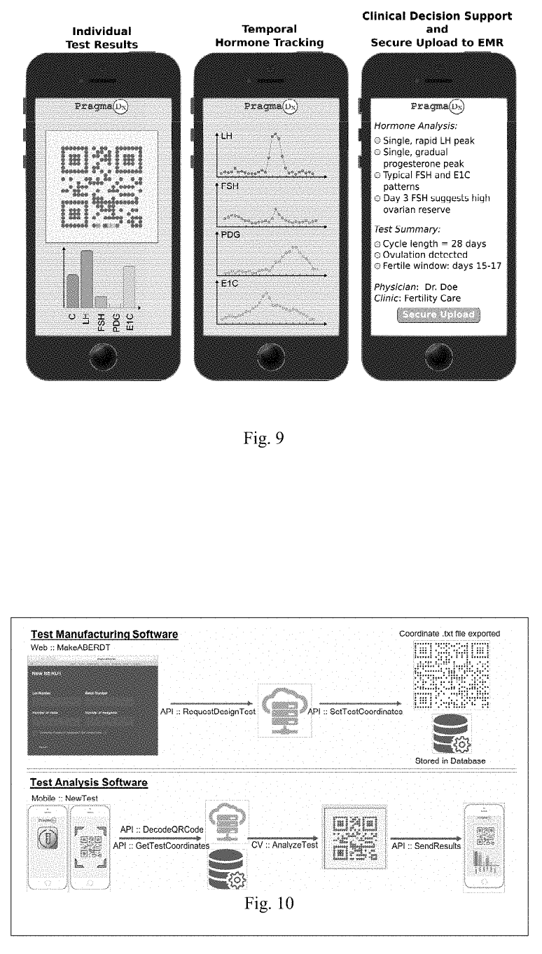

Also provided is the mobile and server-side software for complete analysis (FIG. 9). This software determines the target biomarker concentrations from the automatically captured image of the barcode test. It can track these results over time, and can provide clinical decision support to the patient and their physician. Machine learning algorithms are implemented to classify physiological states depending on biomarker concentrations (e.g., with infertility hormones). A communication system is provided within the mobile application and server-based software that allows patient-physician messaging, as well as secure sharing of test results and test history through the platform.

FIG. 10 is a schematic view of the software architecture for manufacturing beRDTs configured as described herein and analyzing them after administering a test. A user can log into a remote server, for example through a web portal, to a web page that allows the user to engage the test manufacturing software, and to enter information specific to the diagnostic tests that they are making. Such information will typically, but not exclusively, include lot numbers, batch numbers, test numbers, expiration dates, the number of reagents that are to be used per test, and the number of tests to be made in that particular batch. This could also include information about the reagents that are specific to the test, such as (by way of non-limiting example) the names of the analytes that are to be measured.

The foregoing information is then compared to existing entries in the secure, remote database where it is determined if there is a duplicate. In the event that the newly created record is a duplicate, an error message is returned to the user specifying exactly which piece of information was previously existing in the database and caused the error. With this pre-check, the system administrator may be certain that any new test that is created can be uniquely identified within the database. During this process, a unique cryptographic hash may optionally be generated and used to store each unique test's information in the database, as discussed in greater detail below.

If the information that has been input by the user is not a duplicate of a record already in the database, the information is processed by a software implemented rapid diagnostic test creation engine that is responsible for generating the coordinates of the various modules in the barcode. To this end, the test manufacturing software maintains a grid of modules that form the barcode that is to be implemented on a test, such as the grid of modules that form a standard QR code, which forms the template for the creation of test-specific QR codes. Such template includes X-Y coordinates for machine read modules, which are those minimum modules that must be included (i.e., dark modules on a QR code) in order to enable machine reading of the code with an imaging device. The template also includes X-Y coordinates for manufacturing data modules, which are those modules that encode an identification of at least the lot number, batch number, and test number. The test manufacturing software then automatically generates the X-Y coordinates for a complete machine-readable barcode, such as a QR code, that contains this data payload (i.e., at least the machine read modules and the manufacturing data modules identifying a lot number, batch number, and test number), with the test modules randomly assigned to available X-Y coordinates in the grid (i.e., assigned to “open” modules that are neither machine read modules nor manufacturing data modules as described herein, and that may be made “dark” without rendering the resulting QR code non-machine readable). For every target analyte, the software chooses a random module within a specified “test zone.” The modules of the barcode are digitized into a series of “on”/“off” coordinates—here the “on” coordinates represent locations where a reagent should be placed (i.e., the locations of the modules in a physical space). The test module coordinates, once designated, are tested to ensure that they do not destroy the “readability” of the machine-readable barcode. The test zone is likewise selected so as to not destroy the machine-readability of the barcode. Those skilled in the art will recognize that the conversion of data into a machine-readable barcode may be accomplished using ISO standard algorithms.

The test manufacturing software then exports a data file consisting of a text file for each reagent that specifies X-Y coordinates for reagent deposition. If the software chooses a module that was previously “off” or “light” for a target analyte location, it flips this location to an “on” or “dark” location (i.e., it adds an entry into the list of coordinates for the target reagent). If the software chooses a module that was previously “on” or “dark” (i.e., it was going to be printed with a control reagent or otherwise used for the bulk of the machine-readable barcode before being randomly selected as a test module), the test manufacturing software removes it from the control reagent coordinate list and adds it to the target analytes list. This ensures that a given module can only be used for one reagent, and that there are no duplicates. After all test reagents have been assigned locations, the resulting coordinates are verified using ISO standard barcode reading algorithms to verify that the barcode remains machine-readable.

To go from “digital” space (i.e., a matrix or grid of “on” and “off” locations) to “physical” space (i.e., locations on a membrane where reagents can be deposited), each reagent list may be scaled by a parameter that represents the distance between two immediately neighboring modules. A value for this parameter may be empirically determined for various reagents and membranes. Further, each test is spaced by another parameter to allow a gap between each test manufactured. The result of these two steps is a list of X-Y coordinates, in physical space, that would result in a series of machine-readable barcodes if a reagent was placed at each of those coordinates. These results can then be parsed into separate lists for each reagent to allow high-throughput manufacturing.

The test coordinates are stored in the remote, secure database in communication with the test manufacturing software, which coordinates are associated with the lot, batch, and test number.

After administering a test using a test device that has been provided in accordance with the above, a user engages mobile software on a mobile imaging device, such as software on a mobile phone equipped with a camera, which software preferably scans the test barcode, verifies that the test was properly made (i.e., at least is appropriately machine readable to enable reading of data embedded in the barcode), identifies the user responsible for manufacturing the test, decodes the lot number, batch number, and test number from the QR code, and queries the remote, secure database to find the specific X-Y coordinates for each test module on the respective test device. The mobile software then uses this information to analyze the specific test.

A system configured as above allows for the tracking of: (i) which user designed any test; (ii) which user manufactured any test; (iii) linkage of test design, test manufacture, sales, and end use data; and (iv) real-time analysis of each type of test manufactured, with lot-to-lot variation. Such a system, with little modification, may be used by multiple entities in a “white label” setup, in which Entity A would have detailed analysis of each of their tests that was designed/manufactured/used, and Entity B would have a similar analysis for its own tests.

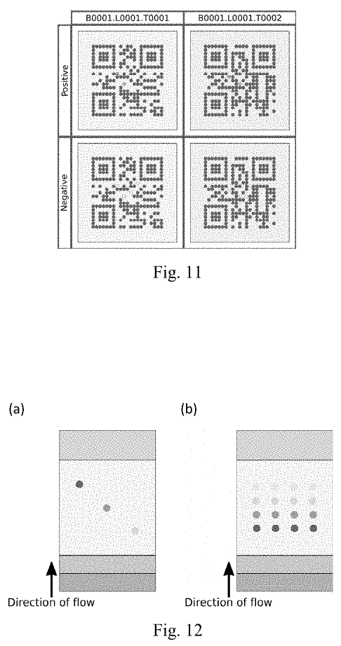

FIG. 11 is a schematic view of various exemplary images that may be produced from the mobile software when testing for various analytes, and particularly shows that small changes in embedded data payload can create drastic changes in barcode appearance, along with the differences between positive and negative results, which adds to the data security benefits that may be achieved through implementation of certain aspects of an embodiment. Significantly, two-dimensional barcodes have built-in error correction of up to 30%. In practice this means that up to 30% of modules can be inverted and the barcode will still be recognizable through automated image processing. This feature is used in implementations of the invention to enhance the privacy and security of the test device. With continuing reference to FIG. 11, the test modules are randomly placed at the time of test design, either taking the place of an existing dark module, or converting a light module to a dark module. When the device is used with a sample, if the target analyte for a particular module is present in the sample, that module will be positive. There are two tests shown in FIG. 11 (“B0001.L0001.T0001” and “B0001.L0001.T0002”), each with four target analytes. The embedded payload within the QR-code shown in FIG. 11 only changes in the last character, but it is clear that this creates a substantial change in module layout within the QR-code. Thus, while QR-codes are readily detected by mobile devices, it is not practical to decode and analyze manually—unlike traditional lateral flow assays that present patient-sensitive results to anyone with a clear view. Results presented in this fashion are private and can only be interpreted by software that has access to the test module locations, stored in a remote database. Importantly, the barcode still scans regardless of the presence of analytes in the sample—this allows the software to identify the test module locations and quantify the amount, or lack thereof, of target analyte. Because the location of these test modules are “hidden” to the user and only interpretable with access to the secure, remote database, mobile and server-side access restrictions allow only the intended users to access the test results. In other words, the test modules are randomly located somewhere within the barcode, but only the secure, remote database has those locations. As a result privacy and security of the test data are significantly improved over previously known rapid diagnostic tests, and test devices configured as discussed above provide significantly improved security over sensitive patient results, particularly useful for stigmatized healthcare conditions, as well as significant resistance against tampering (i.e., a user cannot modify the test to achieve a certain result, simply because they will not know where the test modules are located).

Further, even greater security of test data may be obtained by, instead of particularly encoding the specific lot number, batch number, and test number in the barcode, alternatively generating at the test manufacturing software a cryptographic hash using any standard hashing function (e.g., SHA256) that encodes such information into a fixed length string. This hash is then stored in the secure, remote database and associated with the foregoing manufacturing data, and the secure hash is then embedded as the data payload of the barcode. From this point, the previously described methods for identifying test module locations is followed. This more secure method has the additional benefit of using a cryptographic hash, which is non-sensical until it is decrypted. Thus, through use of such a cryptographic hash, even the test manufacturing details are hidden from the user. Even the slightest variation in input to a cryptographic hash function will result in an entirely different cryptographic hash output. Such secure implementation of the system and method of the invention may have wide-ranging implications, ranging from maintaining confidentiality when testing for stigmatized infectious disease diagnostics (where users would not want others to know what their test was for, let alone the result of the test), to providing general security that would encrypt diagnostic test results as they are wirelessly transmitted.

Those skilled in the art will recognize that alternative unique identifiers may be encoded in the barcode instead of a cryptographic hash and linked with particular manufacturing information as described above without departing from the spirit and scope of the invention.

In accordance with still further aspects of an embodiment of the invention, at least a portion of the barcode, and particularly at least the machine read modules of the barcode, and optionally the manufacturing data modules, may be printed using non-reactive colorimetric reagents that adsorb to the membrane. More particularly, test devices constructed as described above include test devices in which test modules are printed with a specific antibody against a target analyte, and control modules (i.e., the bulk of the machine-readable barcode) were printed with a secondary antibody that captures any excess detection particles. After those reagents are printed, the solvent would evaporate and the antibody would adsorb to the surface of the membrane—but nothing would be visible on the membrane. Only when a solution containing detection nanoparticles would be applied to the beRDT would the machine-readable barcode be visible. In this alternative configuration, rather than printing a secondary antibody for the bulk of the barcode, non-reactive colorimetric reagents that adsorb to the membrane may be used. This allows a user to visualize the machine-readable barcode before the test is used. In addition, in such configuration, secondary antibodies that were previously used to form the bulk of the barcode may instead be used as test control modules—even in the event of a negative test (i.e., no target analyte present), these specific control modules will still bind the detection nanoparticles to inform the user that the test has been properly run. The mobile image capture and analysis software may look for these control modules first, before trying to analyze the test modules in the captured image of the test device. This configuration offers the additional benefit of enabling the user to observe any potential manufacturing defects as they occur, such that any defective tests (e.g., if reagents are incorrectly prepared, it may result in a defective barcode that is not scannable) can be identified and removed before being used by consumers.

Even further and to ensure proper manufacture of test devices before their use, test module reagents may optionally be doped with non-binding, non-interfering colorimetric agents. This allows a user to visualize that the reagent was indeed deposited in the correct location, and avoid any manufacturing errors (e.g., missing test modules, incorrect test module locations, etc.). Because these agents are non-binding, they do not adsorb to the membrane surface, and they are “washed” downstream away from the barcode when a sample is applied. This leaves only the bulk of the barcode that is printed with a non-reactive but membrane-adsorbing colorimetric reagent, and the test modules that are printed with a specific antibody.

Various processes may be implemented to ensure data readability and fidelity in test devices configured as discussed herein. More particularly, lateral flow assays, in general, operate by a fluid being “wicked” down a porous material by capillary action; it is a well-described physical phenomenon. The fluid moves with a slower velocity as it wicks further down the membrane of a lateral flow assay. In other words, it takes longer for the fluid to travel, for example, from 10 mm to 15 mm downstream then it did for the fluid to travel from 5 mm to 10 mm downstream. Even though the distances are the same, the location affects how long it takes for the fluid to travel.

If fluid containing target analytes takes a longer time to flow when it is further downstream, then reagents that are adsorbed to the membrane further downstream will have a longer time to interact with the analyte as it flows past. On an individual analyte level, this increased residence time increases the likelihood of the analyte binding to the capture reagent. On an aggregate level, more analytes binding to the capture reagents at a test module will result in a darker test module, as shown in FIG. 12. Specifically, in FIG. 12(a), with all other parameters being equal, placing test modules further downstream results in a darker colorimetric intensity. With knowledge of these factors, the results for a particular test module on a test device configured as described herein may be adjusted based on the downstream location of that particular module on the membrane.

Because fluid is wicked laminarly (i.e., in straight lines) down the membrane, this behavior changes in the event that test modules are directly in front of one another. As shown in FIG. 12(b), if test modules are placed immediately downstream from each other, the laminar flow results in a depletion of available detection nanoparticles, and therefore, a decrease in colorimetric intensity at the downstream test modules. As there are a limited supply of detection particles, these particles bind to the first test location they come into contact with—typically the upstream test modules. Because they are bound, they are no longer available for binding to downstream test modules. This depleted concentration of detection particles results in a colorimetric gradient across a given column of test modules—the most upstream modules being the darkest, and the most downstream modules being the lightest.

Given the opposite end results depicted in FIGS. 12(a) and 12(b) and described above, these two physical phenomenon (the decreasing speed as fluid moves further down the membrane that results in more binding vs. the higher available concentration of detection nanoparticles for the upstream test modules) can be used to “correct” (or adjust) the test module results. Because the locations of test modules are known at the time of manufacture, we also know their relative upstream/downstream locations and if there are any test modules in front of them that would consume detection nanoparticles, thus enabling a test device specific calibration based on the particular arrangement of modules on the particular barcode.

Further, a testing device configured as described herein may implement internal replicate testing to even further ensure data fidelity. More particularly, if the capture reagent for one of the test modules is printed in multiple locations within the barcode, this allows for internal replicates. The test locations can be stored in the secure, remote database as discussed above, and queried just like other regular test modules. Multiple replicates can be used to: (i) ensure test validity, particularly if the quantification methods detailed previously are used; (ii) ensure that image/environmental artifacts do not negatively affect the image processing; and (iii) help adjust for uneven illumination. With respect to (i), the test modules could be included, by way of non-limiting example, in triplicate. If these modules were placed at different downstream locations within the barcode, the results could be validated by ensuring that the modules agree with each other based on their location (e.g., a test module with a low colorimetric intensity near the leading, upstream modules of the barcode could be expected to have a darker colorimetric intensity if it were placed at a downstream module; placing test modules in different locations and using the quantification methods above would allow for higher degrees of confidence in the accuracy of the result). With respect to (ii), if dust particulates or scratches obscured a particular location on the test or the mobile device's camera, it is possible that the barcode would scan, but that the artifacts might negatively affect the image processing algorithms used to analyze the test result. Having internal replicates in different locations would minimize that risk.

beRDTs, and the associated software, are highly adaptable to different medical conditions and test formats. FIG. 13 shows configurations for various rapid diagnostic tests that may be provided for diagnosing the presence of a wide variety of analytes. For example, FIG. 13(a) provides a remote pregnancy monitoring panel, FIG. 13(b) provides a common blood-borne infection panel, and FIG. 13(c) provides a panel to differentially diagnose between bacterial and viral infections, all of which panels take advantage of the ability of the system and method described herein to analyze multiple analytes from the same sample.

A platform configured as described herein can be used for initial diagnosis, as well as treatment monitoring of infectious diseases, and for chronic disease management. The inventors herein envisage that additional features and functions may be included in the systems and methods disclosed herein, including:
Alternative barcode patterns and registration features. It is possible to capture similar information without the barcode. This would include proprietary shapes and patterns that are identified and decoded using internal software.
New biochemical assays to broaden the applicability of the platform. The list of potential biochemical targets that this could be applied to is extensive, and includes acute care scenarios, infectious diseases, chronic condition monitoring, and monitoring of treatment/disease progression.
A security key embedded within the barcode data payload. This key could be a personalized, one-time use key to allow secure transmission of test results to electronic medical records.
Multi-color tests that use signaling particles of different colors to identify registration features, other analytes to measure, and computer imaging control signals.
Machine-learning algorithms that analyze the multiplexed biomarker barcode embedded rapid diagnostic tests and provide clinical decision support to physicians. These algorithms could provide insight on the likelihood of a particular disease state, or the efficacy of a potential treatment strategy.
Mobile health intervention strategies that improve end-user behavior and lead to healthier outcomes.
Integration with more application programming interfaces, including publicly available APIs for public health (clinic/physician/resource locations), and private APIs for integration into existing telehealth and mobile health infrastructure (including electronic health records and telemedicine software platforms).

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. For example, while the above description primarily focuses on using machine-readable barcodes, it is likewise envisaged that other types of information may be digitized (e.g., converted to X-Y coordinates). For example, a digital image may be converted to a series of X-Y coordinates for reagent deposition, which could be used to create coordinates for a variety of features, including bio-reactive watermarks or proprietary spatial patterns. Likewise, barcode configurations other than QR-codes, such as a DataMatrix code, may similarly be employed using the general concepts and methods described herein. Moreover, in the instance of an image as an input, the coordinates could be generated by any image-processing procedures, including thresholding, edge detection, feature matching, etc., to allow for a variety of outputs from the same input. Thus, it should be understood, therefore, that the invention may be practiced otherwise than as specifically set forth herein.

What is claimed is:

1. A system for determining the presence of a target analyte in a sample, comprising:
a barcode creation software application executable on a server computer and having computer executable instructions configured to:
generate a barcode template including (i) machine read modules designating X-Y coordinates of a first group of said modules necessary for enabling machine reading of said barcode with an imaging device, and (ii) manufacturing data modules designating X-Y coordinates of a second group of said modules encoding manufacturing data relating to a rapid diagnostic test; and
randomly select from said grid of modules X-Y coordinates of a third group of said modules for use as test modules printed with a target molecular recognition element against said target analyte;
a database in data communication with said server computer storing said barcode template;
a rapid diagnostic test device comprising a substrate and a barcode printed from said barcode template on said substrate, said barcode comprised of machine read modules printed at said X-Y coordinates of said first group of said modules, manufacturing data modules printed at said X-Y coordinates of said second group of said modules, and test modules printed at said X-Y coordinates of said third group of modules; and
a computer software application executable on said server computer and configured for data communication with a mobile computing and imaging device, said computer software application having computer executable instructions configured to:
receive an image of said barcode on said rapid diagnostic test device from a mobile computing and imaging device;
analyze said X-Y coordinates of said third group of modules to detect the presence of said analyte;
generate a diagnosis of the presence of said analyte; and
transmit said diagnosis to said mobile computing and imaging device.

2. The system of claim 1, said barcode creation software application further comprising computer executable instructions configured to receive user input of a lot number, a batch number, and a test number, all for association with said test device in said database.

3. The system of claim 2, said barcode creation software application further comprising computer executable instructions configured to receive user input of a number of reagents to be used per test, and a number of tests to be made in a batch, all for association with said test device in said database.

4. The system of claim 1, wherein said database links said rapid diagnostic test device with a test identification number, a lot number, and a batch number and associates said rapid diagnostic test device with a specific end user.

5. The system of claim 1, wherein said manufacturing data modules printed on said rapid diagnostic test device are printed from a control reagent comprising secondary antibodies different from said target molecular recognition element and configured to capture excess detection particles in said test device.

6. The system of claim 5, wherein said machine read modules printed on said rapid diagnostic test device are printed from a control reagent comprising secondary antibodies different from said target molecular recognition element and configured to capture excess detection particles in said test device.

7. The system of claim 5, wherein said machine read modules printed on said rapid diagnostic test device are printed from non-reactive colorimetric reagent.

8. The system of claim 1, wherein said test modules printed on said rapid diagnostic test device are printed from a target molecular recognition element against said target analyte.

9. The system of claim 8, wherein said test modules printed on said rapid diagnostic test device are doped with non-binding, non-interfering colorimetric agents.

10. The system of claim 9, wherein said non-binding, non-interfering colorimetric agents are configured to wash downstream from said barcode on said rapid diagnostic test device upon application of a test sample to said rapid diagnostic test device.

11. The system of claim 1, wherein said manufacturing data modules printed on said rapid diagnostic test device comprise portions of said barcode encoding data representative of a lot number, batch number, and test identification number specific to said rapid diagnostic test device.

12. The system of claim 1, wherein said manufacturing data modules printed on said rapid diagnostic test device comprise portions of said barcode encoding data representative of a unique identifier, wherein said database stores said unique identifier and associates said unique identifier with a lot number, batch number, and test identification number specific to said rapid diagnostic test device.

13. The system of claim 1, wherein said computer software application further comprises computer executable instructions configured to determine a concentration of a target biomarker in said analyte.

14. The system of claim 1, wherein said computer software application further comprises computer executable instructions configured to receive data from said mobile computing and imaging device comprising patient demographic information and patient symptoms.

15. The system of claim 1, wherein said computer software application further comprises computer executable instructions configured to adjust test results based on a downstream location of said modules.

16. The system of claim 1, wherein said computer software application further comprises computer executable instructions configured to adjust test results based on relative positions of multiple of said modules.

17. A method for determining the presence of a target analyte in a sample, comprising:
generating a barcode template at a server computer, said barcode template including (i) machine read modules designating X-Y coordinates of a first group of said modules necessary for enabling machine reading of said barcode with an imaging device, and (ii) manufacturing data modules designating X-Y coordinates of a second group of said modules encoding manufacturing data relating to a rapid diagnostic test;
causing said server computer to randomly select from said grid of modules X-Y coordinates of a third group of said modules for use as test modules printed with a target molecular recognition element against said target analyte;
storing said barcode template in a database in data communication with said server;
providing a rapid diagnostic test device comprising a substrate and a barcode printed from said barcode template on said substrate, said barcode comprised of machine read modules printed at said X-Y coordinates of said first group of said modules, manufacturing data modules printed at said X-Y coordinates of said second group of said modules, and test modules printed at said X-Y coordinates of said third group of modules;
receiving at said server computer from a mobile computing and imaging device an image of said barcode on said rapid diagnostic test device;
causing said server computer to analyze said X-Y coordinates of said third group of modules to detect the presence of said analyte; and
transmitting from said server computer to said mobile computing and imaging device a diagnosis of the presence of said analyte.

18. The method of claim 17, wherein said manufacturing data modules printed on said rapid diagnostic test device are printed from a control reagent comprising secondary antibodies different from said target molecular recognition element and configured to capture excess detection particles in said test device.

19. The method of claim 18, wherein said machine read modules printed on said rapid diagnostic test device are printed from a control reagent comprising secondary antibodies different from said target molecular recognition element and configured to capture excess detection particles in said test device.

20. The method of claim 18, wherein said machine read modules printed on said rapid diagnostic test device are printed from non-reactive colorimetric reagent.

21. The method of claim 17, wherein said test modules printed on said rapid diagnostic test device are printed from a target molecular recognition element against said target analyte.

22. The method of claim 17, wherein said manufacturing data modules printed on said rapid diagnostic test device comprise portions of said barcode encoding data representative of a lot number, batch number, and test identification number specific to said rapid diagnostic test device.

23. The method of claim 17, wherein said manufacturing data modules printed on said rapid diagnostic test device comprise portions of said barcode encoding data representative of a unique identifier, wherein said database stores said unique identifier and associates said unique identifier with a lot number, batch number, and test identification number specific to said rapid diagnostic test device.

24. A method of manufacturing a rapid diagnostic test device for determining the presence of a target analyte in a sample, comprising:
generating a barcode template at a server computer, said barcode template including (i) machine read modules designating X-Y coordinates of a first group of said modules necessary for enabling machine reading of said barcode with an imaging device, and (ii) manufacturing data modules designating X-Y coordinates of a second group of said modules encoding manufacturing data relating to a rapid diagnostic test;
causing said server computer to randomly select from said grid of modules X-Y coordinates of a third group of said modules for use as test modules printed with a target molecular recognition element against said target analyte;
storing said barcode template in a database in data communication with said server; and
forming a rapid diagnostic test device comprising a substrate and a barcode printed from said barcode template on said substrate, said barcode comprised of machine read modules printed at said X-Y coordinates of said first group of said modules, manufacturing data modules printed at said X-Y coordinates of said second group of said modules, and test modules printed at said X-Y coordinates of said third group of modules.

25. The method of claim 24, wherein said manufacturing data modules printed on said rapid diagnostic test device are printed from a control reagent comprising secondary antibodies different from said target molecular recognition element and configured to capture excess detection particles in said test device.

26. The method of claim 25, wherein said machine read modules printed on said rapid diagnostic test device are printed from a control reagent comprising secondary antibodies different from said target molecular recognition element and configured to capture excess detection particles in said test device.

27. The method of claim 25, wherein said machine read modules printed on said rapid diagnostic test device are printed from non-reactive colorimetric reagent.

28. The method of claim 24, wherein said test modules printed on said rapid diagnostic test device are printed from a target molecular recognition element against said target analyte.

29. The method of claim 24, wherein said manufacturing data modules printed on said rapid diagnostic test device comprise portions of said barcode encoding data representative of a lot number, batch number, and test identification number specific to said rapid diagnostic test device.

30. The method of claim 24, wherein said manufacturing data modules printed on said rapid diagnostic test device comprise portions of said barcode encoding data representative of a unique identifier, wherein said database stores said unique identifier and associates said unique identifier with a lot number, batch number, and test identification number specific to said rapid diagnostic test device.
